(19) United States
(12) Patent Application Publication
(10) Pub. No.: US 2025/0134571 A1
(43) Pub. Date: May 1, 2025

(54) SYSTEMS AND METHODS FOR REDUCING MICROBUBBLES IN ELECTROPORATION APPLICATIONS

(71) Applicant: St. Jude Medical, Cardiology Division, Inc., St. Paul, MN (US)

(72) Inventors: Lakshya Mittal, Dayton, MN (US); Jacob J. Daly, Ham Lake, MN (US); Loell B. Moon, Ham Lake, MN (US); Jeffrey M. Fish, Maple Grove, MN (US)

(21) Appl. No.: 18/922,642

(22) Filed: Oct. 22, 2024

Related U.S. Application Data
(60) Provisional application No. 63/545,657, filed on Oct. 25, 2023.

(51) Int. Cl. A61B 18/00

(52) U.S. Cl. CPC A61B 18/00; A61B 2018/00577; A61B 2018/00613; A61B 2018/00761; A61B 2018/00767

(57) ABSTRACT

An electroporation system is provided. The electroporation system includes a catheter comprising a plurality of electrodes, and a pulse generator coupled to the catheter, the pulse generator configured to generate a waveform to be delivered using at least one of the plurality of electrodes. The waveform includes a plurality of bursts, each burst including a plurality of loops, and each loop including a plurality of pulses, wherein each of the plurality of pulses has a pulse width of 3 microseconds (μs) or less, wherein each burst includes no more than ten loops, wherein the plurality of bursts include at least ten bursts, and wherein the pulse widths, number of loops per burst, and number of bursts facilitate reducing microbubble formation.

### CROSS REFERENCE TO RELATED APPLICATIONS

This application claims priority to U.S. Provisional Patent Application No. 63/545,657 filed on Oct. 25, 2023, which is incorporated by reference herein in its entirety.

### FIELD OF THE DISCLOSURE

The present disclosure relates generally to tissue ablation systems. In particular, the present disclosure relates to applying electroporation therapy using waveforms that facilitate reducing microbubble formation.

### BACKGROUND

It is generally known that ablation therapy may be used to treat various conditions afflicting the human anatomy. For example, ablation therapy may be used in the treatment of atrial arrhythmias. When tissue is ablated, or at least subjected to ablative energy generated by an ablation generator and delivered by an ablation catheter, lesions form in the tissue. Electrodes mounted on or in ablation catheters are used to cause tissue destruction in cardiac tissue to correct conditions such as ventricular and atrial arrhythmias (including, but not limited to, ectopic atrial tachycardia, atrial fibrillation, and atrial flutter).

Arrhythmia (i.e., irregular heart rhythm) can create a variety of dangerous conditions including loss of synchronous atrioventricular contractions and stasis of blood flow which can lead to a variety of ailments and even death. It is believed that the primary cause of atrial arrhythmia is stray electrical signals within the left or right atrium of the heart. The ablation catheter imparts ablative energy (e.g., radiofrequency energy, cryoablation, lasers, chemicals, high-intensity focused ultrasound, etc.) to cardiac tissue to create a lesion in the cardiac tissue. This lesion disrupts undesirable electrical pathways and thereby limits or prevents stray electrical signals that lead to arrhythmias.

Electroporation is a substantially non-thermal ablation technique that involves applying strong electric-fields that induce pore formation in the cellular membrane. The electric field may be induced by applying a relatively short duration pulse which may last, for instance, from a nanosecond to several milliseconds. Such a pulse may be repeated to form a pulse train. When such an electric field is applied to tissue in an in vivo setting, the cells in the tissue are subjected to an increased trans-membrane potential, which opens the pores on the cell plasma membrane. Electroporation may be reversible (i.e., the temporally-opened pores will reseal) or irreversible (i.e., the pores will remain open). For example, in the field of gene therapy, reversible electroporation (i.e., temporarily open pores) is used to transfect high molecular weight therapeutic vectors into the cells. In other therapeutic applications, a suitably configured pulse train alone may be used to cause cell destruction, for instance by causing irreversible electroporation.

For example, pulsed field ablation (PFA) may be used to perform instantaneous pulmonary vein isolation (PVI). PFA generally involves delivering high voltage pulses from electrodes disposed on a catheter. For example, voltage pulses may range from less than about 500 volts to about 2400 volts or higher. These fields may be applied between pairs of electrodes (bipolar therapy) or between one or more electrodes and a return patch (monopolar therapy).

In PFA, different waveforms may be used to achieve different goals. For example, some waveforms may result in larger or smaller lesion size than other waveforms. Further, some waveforms result in higher or lower overall energy delivery than other waveforms (less overall energy delivery generally corresponds to less heating of the target tissue). As another example, some waveforms are more likely to induce muscular contractions in a patient. Generally, it is desirable to deliver electroporation therapy with a relatively low number of therapy applications over a relatively short timeframe. Further, it is generally desirable to avoid thermal heating of the tissue, and to have little to no skeletal muscle recruitment (i.e., avoiding muscle contractions). In addition, it is also generally desirable to reduce the likelihood of waveforms generating sustained atrial arrhythmias.

Because it involves application of electrical pulses in a blood pool, PFA for PVI (or other applications) may, in some instances, induce microbubble formation. Microbubble formation may be attributable to, for example, a combination of electrolysis and gas displacement due to shock waves. Microbubbles are generally undesirable. Accordingly, it would be desirable to reduce or eliminate microbubble formation in PFA applications.

### BRIEF SUMMARY OF THE DISCLOSURE

In one aspect, an electroporation system is provided. The electroporation system includes a catheter comprising a plurality of electrodes, and a pulse generator coupled to the catheter, the pulse generator configured to generate a waveform to be delivered using at least one of the plurality of electrodes. The waveform includes a plurality of bursts, each burst including a plurality of loops, and each loop including a plurality of pulses, wherein each of the plurality of pulses has a pulse width of 3 microseconds (μs) or less, wherein each burst includes no more than ten loops, wherein the plurality of bursts include at least ten bursts, and wherein the pulse widths, number of loops per burst, and number of bursts facilitate reducing microbubble formation.

In another aspect, a pulse generator for use with an electroporation system is provided. The pulse generator is configured to be coupled to a catheter including a plurality of electrodes and configured to generate a waveform to be delivered using at least one of the plurality of electrodes. The waveform includes a plurality of bursts, each burst including a plurality of loops, and each loop including a plurality of pulses, wherein each of the plurality of pulses has a pulse width of 3 microseconds (μs) or less, wherein each burst includes no more than ten loops, wherein the plurality of bursts include at least ten bursts, and wherein the pulse widths, number of loops per burst, and number of bursts facilitate reducing microbubble formation.

In yet another aspect, an electroporation system is provided. The electroporation system includes a catheter comprising a plurality of electrodes, and a pulse generator coupled to the catheter, the pulse generator configured to generate a waveform to be delivered using at least one of the plurality of electrodes. The waveform includes a first pulse delivered between a first electrode of the plurality of electrodes and a second electrode of the plurality of electrodes. The system further includes a grounding electrode arrangement configured to discharge, from the first and second electrodes, charge accumulated on the first and second electrodes during delivery of the first pulse.

In yet another aspect, an electroporation system is provided. The electroporation system includes a catheter comprising a plurality of electrodes, and a pulse generator coupled to the catheter, the pulse generator configured to generate a waveform to be delivered using at least one of the plurality of electrodes. The waveform includes a plurality of consecutive pulses delivered between a first effective electrode of the plurality of electrodes and a second electrode of the plurality of electrodes, wherein each of the plurality of pulses is a negative pulse, wherein the first effective electrode has a larger surface area than the second electrode, and wherein the first effective electrode is set to a positive polarity.

The foregoing and other aspects, features, details, utilities, and advantages of the present disclosure will be apparent from reading the following description and claims, and from reviewing the accompanying drawings.

### BRIEF DESCRIPTION OF THE DRAWINGS

FIG. 1 is a schematic and block diagram view of a system for electroporation therapy.

FIGS. 2A and 2B are views of one embodiment of a catheter assembly that may be used with the system shown in FIG. 1.

FIGS. 3A-3C are views of alternative embodiments of a catheter assembly that may be used with the system shown in FIG. 1.

FIG. 4 is one embodiment of a waveform that may be delivered using the system shown in FIG. 1.

### DETAILED DESCRIPTION OF THE DISCLOSURE

The present disclosure provides systems and methods for electroporation. An electroporation system includes a catheter comprising a plurality of electrodes, and a pulse generator coupled to the catheter, the pulse generator configured to generate a waveform to be delivered using at least one of the plurality of electrodes. The waveform includes a plurality of bursts, each burst including a plurality of loops, and each loop including a plurality of pulses, wherein each of the plurality of pulses has a pulse width of 3 microseconds (μs) or less, wherein each burst includes no more than ten loops, wherein the plurality of bursts include at least ten bursts, and wherein the pulse widths, number of loops per burst, and number of bursts facilitate reducing microbubble formation.

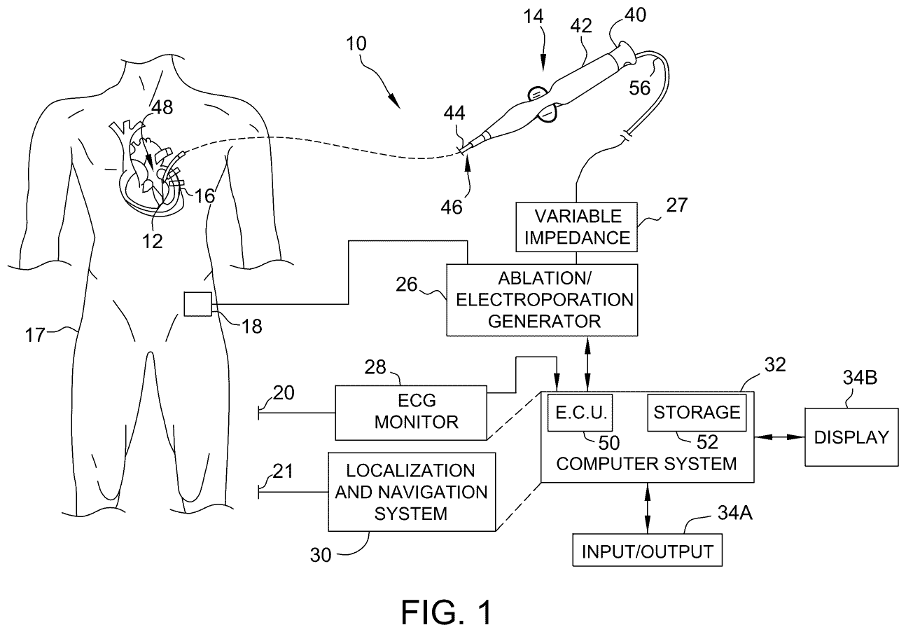

FIG. 1 is a schematic and block diagram view of a system 10 for electroporation therapy. In general, system 10 includes a catheter electrode assembly 12 disposed at a distal end 48 of a catheter 14. As used herein, “proximal” refers to a direction toward the end of the catheter near the clinician and “distal” refers to a direction away from the clinician and (generally) inside the body of a patient. The electrode assembly includes one or more individual, electrically-isolated electrode elements. Each electrode element, also referred to herein as a catheter electrode, is individually wired such that it can be selectively paired or combined with any other electrode element to act as a bipolar or a multi-polar electrode.

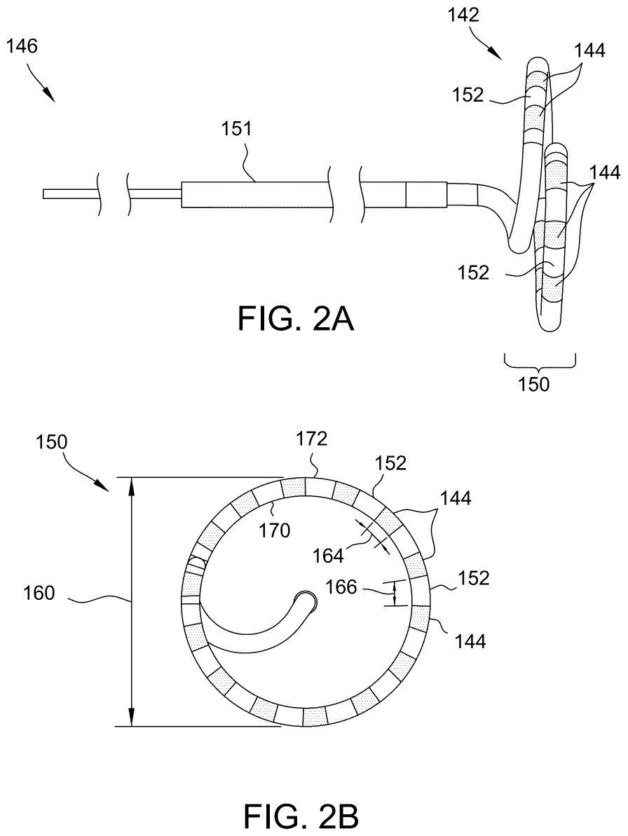

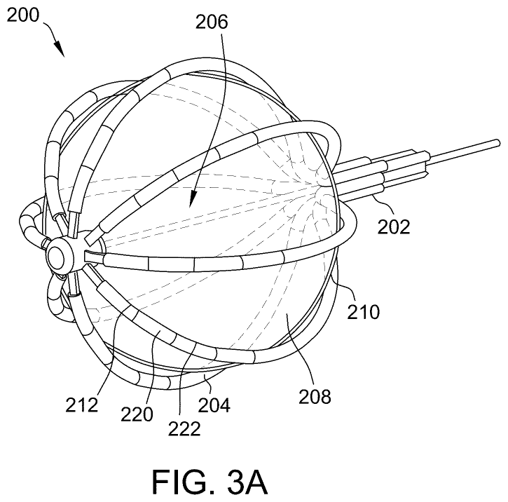

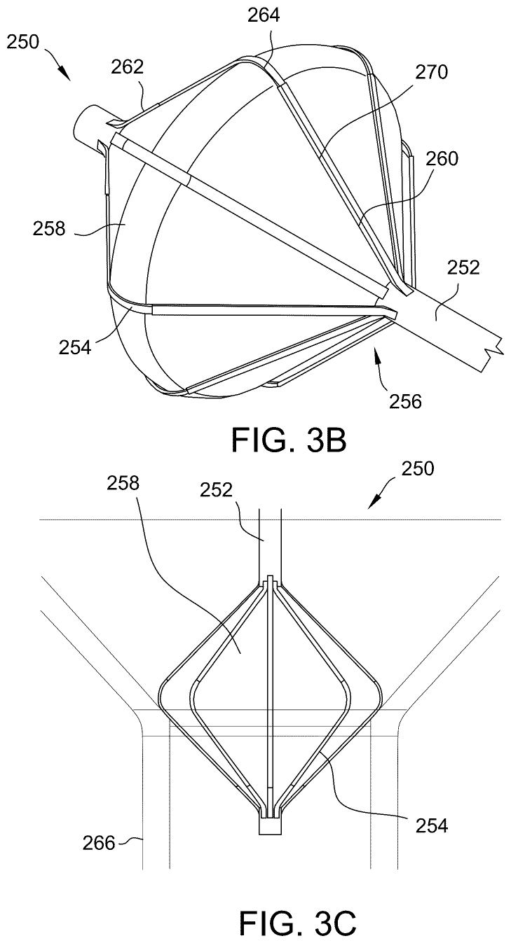

System 10 may be used for irreversible electroporation (IRE) to destroy tissue. In particular, system 10 may be used for electroporation-induced therapy that includes delivering electrical pulses in such a manner as to directly cause an irreversible loss of plasma membrane integrity leading to its breakdown and cell destruction. This mechanism of cell destruction may be viewed as an “outside-in” process, meaning that the disruption of the outside plasma membrane of the cell causes detrimental effects to the inside of the cell. Sometimes these electrical pulses may directly manipulate and damage the intracellular organelles to induce cell death, without causing a significant amount of damage to the cell membrane. Typically, for classical plasma membrane electroporation, electric energy may be delivered as a pulsed electric field in the form of short-duration pulses (e.g., having a 10 nanosecond (ns) to 100 millisecond (ms) duration) between closely spaced electrodes capable of delivering an electric field strength of about 0.05 to 100.0 kilovolts/centimeter (kV/cm). System 10 may be used for high output (e.g., high voltage and/or high current) electroporation procedures. Further, system 10 may be used with a loop catheter such as that depicted in FIGS. 2A and 2B, and/or with a basket catheter such as those depicted in FIGS. 3A-3C. In some embodiments, system 10 is used for reversible electroporation instead of or in addition to irreversible electroporation.

In one embodiment, stimulation is delivered selectively (e.g., between pairs of electrodes) on catheter 14. Further, the electrodes on catheter 14 may be switchable between being connected to a 3D mapping system and being connected to an electroporation generator.

Irreversible electroporation through a multi-electrode catheter may enable pulmonary vein isolation in as few as one shock per vein, which may produce much shorter procedure times compared to sequentially positioning a radiofrequency (RF) ablation tip around a vein. Further, irreversible electroporation may be used for focal ablation procedures. Notably, the embodiments described herein may be used with any suitable irreversible electroporation application.

It should be understood that while the energization strategies are described as involving square wave pulses, embodiments may use variations and remain within the spirit and scope of the disclosure. For example, exponentially-decaying pulses, exponentially-increasing pulses, and combinations may be used.

Further, it should be understood that the mechanism of cell destruction in electroporation is not primarily due to heating effects, but rather to cell membrane disruption (e.g., through pore formation and/or other cell damage) through application of a high-voltage electric field. Thus, electroporation may avoid some possible thermal effects that may occur when using radio frequency (RF) energy. This “colder therapy” thus has desirable characteristics.

With this background, and now referring again to FIG. 1, system 10 includes a catheter electrode assembly 12 including at least one catheter electrode. Electrode assembly 12 is incorporated as part of a medical device such as a catheter 14 for electroporation therapy of tissue 16 in a body 17 of a patient. In the illustrative embodiment, tissue 16 includes heart or cardiac tissue. It should be understood, however, that embodiments may be used to conduct electroporation therapy with respect to a variety of other body tissues (e.g., renal tissue, tumors, etc.).

FIG. 1 further shows a plurality of return electrodes designated 18, 20, and 21, which are diagrammatic of the body connections that may be used by the various sub-systems included in overall system 10, such as an electroporation generator 26, an electrophysiology (EP) monitor such as an ECG monitor 28, and a localization and navigation system 30 for visualization, mapping, and navigation of internal body structures. In the illustrated embodiment, return electrodes 18, 20, and 21 are patch electrodes. It should be understood that the illustration of a single patch electrode is diagrammatic only (for clarity) and that such sub-systems to which these patch electrodes are connected may, and typically will, include more than one patch (body surface) electrode, and may include split patch electrodes (as described herein). Further, in some embodiments, in a multiplexing arrangement, therapy may repeatedly switch between using a different return electrodes 18, 20, 21. In other embodiments, return electrodes 18, 20, and 21 may be any other type of electrode suitable for use as a return electrode including, for example, one or more catheter electrodes. Return electrodes that are catheter electrodes may be part of electrode assembly 12 or part of a separate catheter or device (not shown). System 10 may further include a main computer system 32 (including an electronic control unit 50 and data storage-memory 52), which may be integrated with localization and navigation system 30 in certain embodiments. System 32 may further include conventional interface components, such as various user input/output mechanisms 34A and a display 34B, among other components.

Electroporation generator 26 is configured to energize the electrode element(s) in accordance with an electroporation energization strategy, which may be predetermined or may be user-selectable. For electroporation therapy, generator 26 may be configured to produce an electric energy that is delivered via electrode assembly 12 as a pulsed electric field in the form of short-duration square wave pulses (e.g., a nanosecond to several milliseconds duration, or any duration suitable for electroporation) between closely spaced electrodes capable of delivering an electric field strength (i.e., at the tissue site) of about 0.05 to 100.0 kV/cm. The amplitude and pulse width needed for irreversible electroporation are inversely related. That is, as pulse widths are decreased, the amplitude may generally be increased to achieve chronaxie. The electric energy may be delivered, for example, using a fixed voltage delivery system (in which a fixed voltage is applied, independent of a patient impedance) or using a fixed current delivery system (in which a fixed current is achieved by adjusting the voltage based on the patient impedance). In a fixed current delivery system, the patient impedance may be determined, for example, by delivering a relatively small voltage pulse and measuring current to calculate impedance, or by delivering an AC current waveform and measuring voltage to calculate impedance. A fixed current system may also involve measuring current during therapy delivery and actively adjusting voltage between pulses.

Electroporation generator 26, sometimes also referred to herein as a DC energy source, is a biphasic electroporation generator 26 configured to generate a series of energy pulses that all produce current in two directions (i.e., positive and negative pulses). In other embodiments, electroporation generator is a monophasic or polyphasic electroporation generator. In some embodiments, electroporation generator 26 is configured to output energy in pulses at selectable energy levels, such as fifty joules, one hundred joules, two hundred joules, and the like. Other embodiments may have more, or fewer energy settings and the values of the available setting may be the same or different (settings may include, e.g., waveform parameters, voltage, current, number of applications, etc.). For successful electroporation, some embodiments utilize the two hundred joule output level. For example, electroporation generator 26 may output a pulse having a peak magnitude from about 10 Volts (V) to about 20,000 V. Other embodiments may output any other suitable positive or negative voltage.

In some embodiments, a variable impedance 27 allows the impedance of system 10 to be varied to limit arcing. Moreover, variable impedance 27 may be used to change one or more characteristics, such as amplitude, duration, pulse shape, and the like, of an output of electroporation generator 26. Although illustrated as a separate component, variable impedance 27 may be incorporated in catheter 14 or generator 26.

With continued reference to FIG. 1, as noted above, catheter 14 may include functionality for electroporation and in certain embodiments also additional ablation functions (e.g., RF ablation). It should be understood, however, that in those embodiments, variations are possible as to the type of ablation energy provided (e.g., cryoablation, ultrasound, etc.).

In the illustrative embodiment, catheter 14 includes a cable connector or interface 40, a handle 42, and a shaft 44 having a proximal end 46 and a distal 48 end. Catheter 14 may also include other conventional components not illustrated herein such as a temperature sensor, additional electrodes, and corresponding conductors or leads. Connector 40 provides mechanical and electrical connection(s) for cable 56 extending from generator 26. Connector 40 may include conventional components known in the art and as shown is disposed at the proximal end of catheter 14.

Handle 42 provides a location for the clinician to hold catheter 14 and may further provide means for steering or the guiding shaft 44 within body 17. For example, handle 42 may include means to change the length of a guidewire extending through catheter 14 to distal end 48 of shaft 44 or means to steer shaft 44. Moreover, in some embodiments, handle 42 may be configured to vary the shape, size, and/or orientation of a portion of the catheter, and it will be understood that the construction of handle 42 may vary. In an alternate embodiment, catheter 14 may be robotically driven or controlled. Accordingly, rather than a clinician manipulating a handle to advance/retract and/or steer or guide catheter 14 (and shaft 44 thereof in particular), a robot is used to manipulate catheter 14. Shaft 44 is an elongated, tubular, flexible member configured for movement within body 17. Shaft 44 is configured to support electrode assembly 12 as well as contain associated conductors, and possibly additional electronics used for signal processing or conditioning. Shaft 44 may also permit transport, delivery and/or removal of fluids (including irrigation fluids and bodily fluids), medicines, biologics, and/or surgical tools or instruments. Shaft 44 may be made from conventional materials such as polyurethane and defines one or more lumens configured to house and/or transport electrical conductors, fluids or surgical tools, as described herein. Shaft 44 may be introduced into a blood vessel or other structure within body 17 through a conventional introducer. Shaft 44 may then be advanced/retracted and/or steered or guided through body 17 to a desired location such as the site of tissue 16, including through the use of guidewires or other means known in the art.

Localization and navigation system 30 may be provided for visualization, mapping and navigation of internal body structures. Localization and navigation system 30 may include conventional apparatus known generally in the art. For example, localization and navigation system 30 may be substantially similar to the EnSite Precision™ System, commercially available from Abbott Laboratories, and as generally shown in commonly assigned U.S. Pat. No. 7,263,397 titled “Method and Apparatus for Catheter Navigation and Location and Mapping in the Heart”, the entire disclosure of which is incorporated herein by reference. In another example, localization and navigation system 30 may be substantially similar to the EnSite X™ System, as generally shown in U.S. Pat. App. Pub. No. 2020/0138334 titled “Method for Medical Device Localization Based on Magnetic and Impedance Sensors”, the entire disclosure of which is incorporated herein by reference. It should be understood, however, that localization and navigation system 30 is an example only, and is not limiting in nature. Other technologies for locating/navigating a catheter in space (and for visualization) are known, including for example, the CARTO navigation and location system of Biosense Webster, Inc., the Rhythmia® system of Boston Scientific Scimed, Inc., the KODEX® system of Koninklijke Philips N.V., the AURORA® system of Northern Digital Inc., commonly available fluoroscopy systems, or a magnetic location system such as the gMPS system from Mediguide Ltd.

In this regard, some of the localization, navigation and/or visualization systems may include a sensor for producing signals indicative of catheter location information, and may include, for example, one or more electrodes in the case of an impedance-based localization system, or alternatively, one or more coils (i.e., wire windings) configured to detect one or more characteristics of a magnetic field, for example in the case of a magnetic-field based localization system. As yet another example, system 10 may utilize a combination electric field-based and magnetic field-based system as generally shown with reference to U.S. Pat. No. 7,536,218 entitled “Hybrid Magnetic-Based and Impedance Based Position Sensing,” the disclosure of which is incorporated herein by reference in its entirety.

Pulsed field ablation (PFA), which is a methodology for achieving irreversible electroporation and cell death, may be implemented using the systems and methods described herein. In some cases, PFA may be used at specific cardiac tissue sites such as the pulmonary veins to perform a pulmonary vein isolation (PVI), or to perform focal ablation. In PFA, electric fields may be applied between adjacent electrodes (in a bipolar approach) or between one or more electrodes and a return patch (in a monopolar approach). There are advantages and disadvantages to each of these approaches.

Both approaches, using an appropriate electrode geometry, are able to provide contiguous lesions. For lesion size and proximity, the monopolar approach can potentially create deeper lesions with the same applied voltage. Further, the monopolar approach may be able to create lesions from a distance (e.g., generally proximate, but not necessarily contacting tissue). The bipolar approach may create smaller lesions, requiring closer proximity or contact with tissue to create transmural lesions (depending on, for example, tissue thickness). To monitor operation of system 10, one or more impedances between catheter electrodes 144 and/or return electrodes 18, 20, and 21 may be measured. For example, for system 10, impedances may be measured as described in U.S. Patent Application Publication No. 2019/0117113, filed on Oct. 23, 2018, U.S. Patent Application Publication No. 2019/0183378, filed on Dec. 19, 2018, and U.S. Patent Application No. 63/027,660, filed on May 20, 2020, all of which are incorporated by reference herein in their entirety.

FIGS. 2A and 2B are views of one embodiment of a catheter assembly 146 that may be used with catheter 14 in system 10. Catheter assembly 146 may be referred to as a loop catheter.

Those of skill in the art will appreciate that, in other embodiments, any suitable catheter may be used. That is, the systems and methods described herein are not limited to use with the particular catheter assemblies shown. For example, the systems and methods described herein may be implemented in a linear catheter, a grid catheter (e.g., a catheter including a number of splines arranged in a plane, each spline including one or more electrodes), and/or a focal ablation catheter (e.g., such as the Abbott TactiFlex catheter and TactiCath catheter).

Specifically, FIG. 2A is a side view of catheter assembly 146 with a variable diameter loop 150 at a distal end 142. FIG. 2B is an end view of variable diameter loop 150 of catheter assembly 146. Those of skill in the art will appreciate that the methods and systems described herein may be implemented using any suitable catheter (e.g., fixed loop catheters, linear catheters, basket catheter, etc.). As shown in FIGS. 2A and 2B, variable diameter loop 150 is coupled to a distal section 151 of shaft 44.

Variable diameter loop 150 is selectively transitionable between an expanded (also referred to as “open”) diameter 160 (shown in FIG. 2A) and a retracted (also referred to as “closed”) diameter 160 (not shown). In the example embodiment, an expanded diameter 160 is twenty eight mm and a retracted diameter 160 is fifteen mm. In other embodiments, diameter 160 may be variable between any suitable open and closed diameters 160.

In the embodiment shown, variable diameter loop 150 includes fourteen catheter electrodes 144 substantially evenly spaced around the circumference of variable diameter loop 150 in the expanded configuration. In the retracted configuration, one or more of electrodes 144 may overlap. In other embodiments, other arrangements of catheter electrodes 144 may be implemented. For example, in one embodiment, variable diameter loop 150 includes twelve catheter electrodes 144.

Catheter electrodes 144 are platinum ring electrodes configured to conduct and/or discharge electrical current in the range of one thousand volts and/or ten amperes. In other embodiments, variable diameter loop 150 may include any suitable number of catheter electrodes 144 made of any suitable material. Catheter electrodes 144 may include any catheter electrode suitable to conduct high voltage and/or high current (e.g., in the range of one thousand volts and/or ten amperes). Each catheter electrode 144 is separated from each other catheter electrode by an insulated gap 152. In the example embodiment, each catheter electrode 144 has a same length 164 (shown in FIG. 2B) and each insulated gap 152 has a same length 166 as each other gap 152. Length 164 and length 166 are both about 2.5 mm in the example embodiment. In other embodiments, length 164 and length 166 may be different from each other. Moreover, in some embodiments, catheter electrodes 144 may not all have the same length 164 and/or insulated gaps 152 may not all have the same length 166. In some embodiments, catheter electrodes 144 are not spaced evenly around the circumference of variable diameter loop 150.

Diameter 160 and catheter electrode 144 spacing may be developed to provide a targeted range of energy density to tissue, as well as to provide sufficient electroporation coverage for different human anatomic geometries. In general, a sufficient number of electrodes 144 with appropriate lengths 164 are desired to provide substantially even and continuous coverage around the circumference of variable diameter loop 150, while still allowing enough flexibility to allow variable diameter loop 150 to expand and contract to vary diameter 160 to the desired extremes.

As mentioned above, length 164 of catheter electrodes 144 may be varied. Increasing length 164 of catheter electrodes 144 may increase coverage of electrodes 144 around the circumference of variable diameter loop 150 while also decreasing current density (by increasing the surface area) on electrodes 144, which may help prevent arcing and thermal effects during electroporation operations. Increasing length 164 too much, however, may prevent variable diameter loop 150 from forming a smooth circular shape and may limit the closed diameter 160 of variable diameter loop 150. Additionally, too great a length 164 may increase the surface area of catheter electrodes 144 to a point that the current density applied to catheter electrodes 144 by a power source is below the minimum current density needed for successful therapy. Conversely, decreasing length 164 decreases the surface area, thereby increasing the current density (assuming no other system changes) on catheter electrodes 144. As discussed above, greater current densities may lead to increased risk of arcing and heating during electroporation, and may result in larger additional system resistances needing to be added to prevent arcing. Moreover, in order to get a desired, even coverage about the circumference of variable diameter loop 150, more catheter electrodes 144 may be needed if length 164 is decreased. Increasing the number of catheter electrodes 144 on variable diameter loop 150 may prevent variable diameter loop 150 from being able to be contracted to a desired minimum diameter 160.

FIG. 3A is a perspective view of an alternative catheter assembly 200 that may be used with catheter 14. Catheter assembly 200 may be referred to as a basket catheter. Catheter assembly 200 includes a shaft 202 and a plurality of splines 204 surrounding a distal portion 206 of shaft 202. In this embodiment, catheter assembly 200 also includes a balloon 208 enclosed by splines 204. Balloon 208 may be selectively inflated to fill the space between splines 204. Notably, balloon 208 functions as an insulator, and generally reduces energy losses, which may result in increased lesion size. In some embodiments, balloon 208 may be filled with a cold media (e.g., a cryofluid or cold saline). Further, in some embodiments balloon 208 may be a double-layered balloon.

Each spline 204 includes a proximal end 210 coupled to shaft 202 and a distal end 212 coupled to shaft 202. From proximal end 210 to distal end 212, spline 204 has an arcuate shape that extends radially outward.

In this embodiment, each spline 204 includes a plurality of individual electrodes 220. For example, each spline 204 may include an elastic material (e.g., Nitinol) covered in a polymer tube 222, with individual electrodes 220 attached to an exterior of polymer tube 222. In the embodiment shown, each spline 204 includes two electrodes 220. Further, as shown in FIG. 2, electrodes 220 are generally positioned closer to distal end 212 than proximal end 210 to correspond to portions of spline 204 that will contact the pulmonary vein.

Alternatively, each spline 204 may include any suitable number and arrangement of electrodes 220. For example, in some embodiments, each spline 204 includes four electrodes 220.

In this embodiment, alternating splines 204 alternate polarities. That is, electrodes 220 on a particular spline 204 have the same polarity, but electrodes 220 on a particular spline 204 have a different polarity than electrodes 220 on adjacent splines 204. Alternatively, any suitable polarization scheme may be used. During delivery, splines 204 may be collapsed in towards shaft 202. Subsequently, to perform ablation, splines 204 are deployed to extend radially outward.

Splines 204 may all have the same length, or at least some of splines 204 may have different lengths. Further, insulating material on each spline 204 may have the same length, or at least some splines 204 may have insulating material with different lengths. In addition, in some embodiments, catheter assembly 200 includes a distal electrode (not shown) positioned distal of splines 204. The distal electrode may be used to perform point ablation (e.g., by creating a bipole between the distal electrode and one of splines 204), and/or may be used for visualization/mapping purposes (e.g., using the distal electrode in combination with an electrode on shaft 202).

FIG. 3B is a perspective view of an alternative catheter assembly 250 that may be used with catheter 14, and FIG. 3C is a side schematic view of catheter assembly 250. Like catheter assembly 200 (shown in FIG. 3A), catheter assembly 250 may be referred to as a basket assembly.

Catheter assembly 250 includes a shaft 252 and a plurality of splines 254 surrounding a distal portion 256 of shaft 252. In this embodiment, catheter assembly 250 includes a balloon 258 enclosed by splines 254. Balloon 258 may be selectively inflated to occupy the space between splines 254. Notably, balloon 258 functions as an insulator, and generally reduces energy, which may result in increased lesion size.

Each spline 254 includes a proximal end 260 coupled to shaft 252 and a distal end 262 coupled to shaft 252. From proximal end 260, spline 1004 extends radially outward to an inflection point 264, and then extends radially inward to distal end 262. FIG. 3C shows catheter assembly 250 positioned within the pulmonary vein 266.

A body of each spline 254 is made of an elastic material (e.g., Nitinol), and functions as a relatively large electrode. In this embodiment, alternating splines 254 alternate polarities. That is, each positive spline 254 is positioned between two negative splines 254 and vice-versa. Alternatively, any suitable polarization scheme may be used.

To control the ablation zone of each spline 254, portions of each spline 254 may be covered with insulating material 270 (e.g., heat-shrink or polymer tubing or spray or dip coat with polyimide or PEBAX), and the exposed portions of splines 254 function as electrodes. In the embodiment shown in FIGS. 3B and 3C, inflection point 264 and portions of spline 254 between inflection point 264 and distal end 262 are generally exposed, while portions of spline 254 between inflection point 264 and proximal end 260 are generally insulated. This results in the portions of spline 254 that contact pulmonary vein 266 being exposed (see FIG. 3C). Alternatively, any suitable insulation configuration may be used.

During delivery, splines 254 and balloon 258 may be collapsed. To perform ablation, splines 254 are deployed with inflection points 264 extending radially outward, and balloon 258 is selectively inflated to occupy the space between splines 254.

The combination of balloon 258 and splines 254 facilitates straightforward delivery and deployment of catheter assembly 250. Further, balloon 258 drives more energy into ablated tissue, and stabilizes splines 254 to prevent lateral movement. In addition, using splines 254 as electrodes instead of individual smaller electrodes may facilitate reducing the cost and increasing the reliability of catheter assembly 250.

Splines 254 may all have the same length, or at least some of splines 254 may have different lengths. Further, insulating material 270 on each spline 254 may have the same length, or at least some splines 254 may have insulating material 270 with different lengths. In addition, in some embodiments, catheter assembly 250 includes a distal electrode (not shown) positioned distal of splines 254. The distal electrode may be used to perform point ablation (e.g., by creating a bipole between the distal electrode and one of splines 254), and/or may be used for visualization/mapping purposes (e.g., using the distal electrode in combination with an electrode on shaft 252).

Those of skill the art will appreciate that catheter assembly 146 (shown in FIGS. 2A and 2B), catheter assembly 200 (shown in FIG. 3A), and catheter assembly 250 (shown in FIGS. 3B and 3C) are merely examples. Notably, the systems and methods described herein may be implemented using any suitable catheter assembly.

For electroporation therapy, waveforms are generated using a pulse generator (e.g., electroporation generator 26 (shown in FIG. 1)) and applied between two or more catheter electrodes (i.e., a bipolar approach) or between individual catheter electrodes and a return patch (i.e., a monopolar approach). The waveforms may be monophasic, biphasic (i.e., having both a positive pulse and a negative pulse), or polyphasic. Further, the waveforms may include one or more bursts of pulses (with each burst including multiple pulses). Further, the waveforms are defined by multiple parameters (e.g., pulse width, pulse amplitude, frequency, etc.).

Different waveforms may be used to achieve different goals. For example, some waveforms may result in larger or smaller lesion size than other waveforms. Further, some waveforms result or higher or lower overall energy delivery than other waveforms (less overall energy delivery generally corresponds to less heating of the target tissue). As another example, some waveforms are more likely to induce muscular contractions in a patient. Generally, it is desirable to deliver electroporation therapy with a relatively low number of therapy applications over a relatively short timeframe. Further, it is generally desirable to avoid thermal heating of the tissue, and to have little to no skeletal muscle recruitment (i.e., avoiding muscle contractions). Further, as noted above, it is desirable to reduce or eliminate microbubble formation.

As explained in detail herein, there are a number of different techniques and strategies that may be implemented to facilitate reducing microbubble formation. For example, electrical pulse polarity (e.g., monophasic versus biphasic pulses), pulse width, electrode polarity switching arrangements, electrode multiplexing, number of pulses per burst, grounding electrode arrangements, and/or electrode surface areas may be adjusted or implemented to facilitate reducing microbubble formation. These approaches may be employed alone or in combination with one another to achieve PFA therapy applications that reduce microbubble formation, increase lesion size, reduce thermal output, reduce skeletal muscle recruitment, and reduce total therapy time.

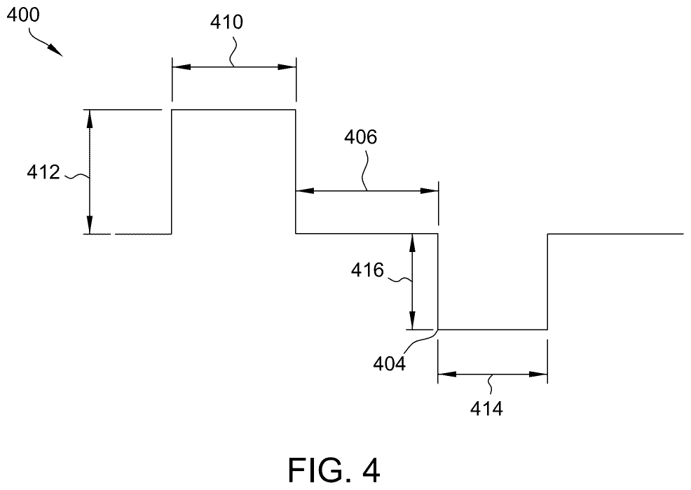

FIG. 4 is one embodiment of a waveform 400. Waveform 400 includes a positive pulse 402 followed by a negative pulse 404. Further, there is an intrapulse delay 406 between the positive and negative pulses 402 and 404.

As shown in FIG. 4, positive pulse 402 has a first pulse width 410 and a first pulse amplitude 412. Similarly, negative pulse 404 has a second pulse width 414 and a second pulse amplitude 416. Waveform 400 may be symmetric (i.e., with first pulse width 410 and first pulse amplitude 412 substantially equal to second pulse width 414 and second pulse amplitude 416) or asymmetric (i.e., with at least one of first pulse width 410 and first pulse amplitude 412 different from second pulse width 414 and second pulse amplitude 416).

When first pulse amplitude 412 and second pulse amplitude 416 are both non-zero, waveform 400 is biphasic (i.e., as shown in FIG. 4). For a monophasic waveform, one of first pulse amplitude 412 and second pulse amplitude 416 is zero. For example, if first pulse amplitude 412 is zero, waveform 400 is monophasic with single negative pulse 404. If second pulse amplitude 416 is zero, waveform 400 is monophasic with single positive pulse 402.

In one biphasic example, first and second pulse widths 410 and 414 may each be 3 microseconds (3 μs), with an intrapulse delay of 1 μs. This may be referred to as a 3-1-3 waveform (i.e., first pulse width of 3 μs—intrapulse delay of 1 μs—second pulse width of 3 μs). First and second pulse amplitudes 412 and 416 may each be, for example, on the order of 1800 Volts (1800V).

In one monophasic example, first pulse width 410 is 0 μs, and second pulse width 414 is 3 μs, with an intrapulse delay of 1 μs. This may be referred to as a 0-1-3 waveform (i.e., first pulse width of 0 μs—intrapulse delay of 1 μs—second pulse width of 3 μs). Second pulse amplitude 416 may be, for example, on the order of 1800V. In another example, the intrapulse delay may be 0 μs.

During an electrode channel window, one or more pulses (e.g., monophasic and/or biphasic waveforms) are delivered by selected electrodes. Those of skill in the art will appreciate that many different electrode energization schemes are possible.

For example, consider catheter assembly 250 (shown in FIGS. 3B and 3C), which includes eight electrodes (with each spline 254 functioning as an electrode). The eight electrodes may be numbered, in order around a circumference of catheter assembly 250, E1, E2, . . . . E8. In one example energization scheme, during a first electrode channel window, a first waveform is delivered between E1 (set to a positive voltage) and E2 (set to a negative voltage), and a second waveform is simultaneously delivered between E5 (set to a positive voltage) and E6 (set to a negative voltage). During a second electrode channel window, a first waveform is delivered between E3 (set to a positive voltage) and E2 (set to a negative voltage), and a second waveform is simultaneously delivered between E7 (set to a positive voltage) and E6 (set to a negative voltage). During a third electrode channel window, a first waveform is delivered between E3 (set to a positive voltage) and E4 (set to a negative voltage), and a second waveform is simultaneously delivered between E7 (set to a positive voltage) and E8 (set to a negative voltage). Further, during a fourth electrode channel window, a first waveform is delivered between E5 (set to a positive voltage) and E4 (set to a negative voltage), and a second waveform is simultaneously delivered between E1 (set to a positive voltage) and E8 (set to a negative voltage). Again, those of skill in the art will appreciate that many different electrode energization schemes are possible.

Multiple electrode channel windows constitute a loop. That loop completes when the pattern of electrode energization begins repeating. For example, a single loop may include the first, second, third, and fourth electrode channel windows described above. Once that loop completes, a subsequent loop would begin, starting again with the first electrode channel window.

Multiple loops constitute a burst, and a therapy session may include one or more bursts. For example, each burst may include one to twenty loops, and a therapy session may include ten to twenty bursts. In general, the period of time between subsequent bursts is longer than the period of time between loops within a burst. That is, i) the time between the end of a last pulse of a last loop in a first burst and the beginning of a first pulse of a first loop in a second, subsequent burst is greater than ii) the time between the end of a last pulse of the second last loop in the first burst and the beginning of a first pulse of the last loop in the first burst.

In one example, each electrode channel window has a length of 60 μs (e.g., using a 3-1-3 waveform plus a 53 us delay), each loop has a length (which may also be referred to as a loop period) of 330 μs (e.g., four 60 us electrode channel windows plus a 90 us delay), and each burst has a length of 3.3 milliseconds (ms) (e.g., ten 330 us loops). Again, these values are only examples, and any suitable time periods may be used.

### Pulse Polarity and Pulse Width

As noted above, electrical pulse polarity (e.g., monophasic versus biphasic pulses) and pulse width may impact microbubble formation. To evaluate this impact, microbubble formation was evaluated for various PFA waveforms, including 3-1-0, 0-1-3, 3-1-3, 1-1-1, and 2-2-2 waveforms. These waveforms were also evaluated at various voltage levels (e.g., 1800V and 2200V).

From this analysis, it was determined that monophasic waveforms (as compared to biphasic waveforms) generally result in significantly higher microbubble volume and number at various pulse widths. For example, monophasic waveforms may result in five to ten times more microbubbles.

Regarding pulse width, similar total bubble volumes were observed for 3-1-3 waveforms at 1800V, 1-1-1 waveforms at 1800V, and 2-1-2 waveforms at 2200V. However, a reduced bubble volume was observed for 1-1-1 waveforms at 2200V. Further, the total bubble numbers were generally reduced for shorter pulse widths. Accordingly, reduced pulse widths appear to significantly reduce microbubble formation. For example, to facilitate reducing microbubble production, in at least some embodiments, a monophasic or biphasic waveform includes pulse widths of 10 us or less, or more particularly 3 us or less, or more particularly 2 us or less, or more particularly 1 μs or less (e.g., including pulse widths of 500 ns or less, 100 ns or less, 10 ns or less, or Ins or less). For example, in some embodiments, a monophasic or biphasic waveform includes pulses in a range from 1 μs to 3 μs.

For monophasic waveforms, the application of multiple pulses results in an accumulation of negative charges on the cathode, and positive charges on the electrode. This charge buildup results in the production of microbubbles, possibly due to electrolysis. In contrast, because biphasic waveforms reverse pulse polarity, the charge accumulation is greatly reduced.

Reducing the intrapulse delay between the positive and negative pulses of a biphasic waveform also facilitates reducing charge buildup, and thus microbubble formation. Further, increasing how often the pulses occur (or reducing pulse width) also reduces charge buildup, which likely explains why reduced pulse widths appear to significantly reduce microbubble formation.

### Number of Pulses Per Burst

As described above, the number of pulses per burst may also impact microbubble formation. To evaluate this impact, microbubble formation was evaluated for various PFA burst patterns. Notably, it was observed that reducing the number of pulses per burst (e.g., by reducing the number of loops per burst) while increasing the total number of bursts, resulted in reduced microbubble formation. That is, spreading out the pulses over a larger number of bursts resulted in reduced microbubble formation. This also facilitated reducing thermal effects, as less pulses are applied over a given time period.

Spreading out the pulses over a larger period of time allows for any accumulated charges on the electrodes to decay and allows for reduced heating. As noted above, reducing charge buildup generally reduces microbubble production. Further, reduced heating may also reduce microbubble production.

As explained above, a loop includes an electrode energization pattern (i.e., including one or more pulses). A new, subsequent loop begins when the electrode energization pattern begins to repeat. Further, multiple loops constitute a burst.

To reduce microbubble formation, the loops may be spread out over multiple bursts. For example, to facilitate reducing microbubble production, in at least some embodiments, a pulse pattern includes at least one burst, or more particularly at least five bursts, or more particularly at least ten bursts, or more particularly at least fifteen bursts, or more particularly at least twenty bursts. Further, in at least some embodiments, each burst includes no more than twenty loops, or more particularly no more than ten loops, or more particularly no more than five loops, or more particularly no more than three loops.

### Electrode Polarity Switching Arrangements

As described above, electrode polarity switching arrangements may also impact microbubble formation. To evaluate this impact, microbubble formation was evaluated for various PFA switching arrangements.

As described above, one energization scheme for catheter assembly 250 (shown in FIGS. 3B and 3C) includes delivering pulses between various pairs of electrodes E1-E8 over four electrode channel windows. In that energization scheme, each electrode is set to the same polarity throughout the energization scheme. For example, E1 is set to a positive voltage when used in combination with E2 (in the first electrode channel window) and when used in combination with E8 (in the fourth electrode channel window). Phrased another way, odd electrodes (E1, E3, E5, and E7) are always set to a positive voltage, and even electrodes (E2, E4, E6, and E8) are always set to a negative voltage. This is referred to herein as a first energization scheme.

In a second, different energization scheme, each electrode switches between operating at a positive voltage and operating at a negative voltage throughout the energization scheme. For example, during a first electrode channel window, a first waveform is delivered between E1 (set to a positive voltage) and E2 (set to a negative voltage), and a second waveform is simultaneously delivered between E5 (set to a positive voltage) and E6 (set to a negative voltage). During a second electrode channel window, a first waveform is delivered between E3 (set to a negative voltage) and E2 (set to a positive voltage), and a second waveform is simultaneously delivered between E7 (set to a negative voltage) and E6 (set to a positive voltage). During a third electrode channel window, a first waveform is delivered between E3 (set to a positive voltage) and E4 (set to a negative voltage), and a second waveform is simultaneously delivered between E7 (set to a negative voltage) and E8 (set to a positive voltage). Further, during a fourth electrode channel window, a first waveform is delivered between E5 (set to a negative voltage) and E4 (set to a positive voltage), and a second waveform is simultaneously delivered between E1 (set to a negative voltage) and E8 (set to a positive voltage). Accordingly, in the second energization scheme, each electrode switches between being set to a positive voltage and being set to a negative voltage.

The first and second energization schemes were evaluated in regards to microbubble formation. Notably, switching the electrode polarity (i.e., as in the second energization scheme) resulted in reduced microbubble formation. Further, microbubble formation was further reduced by spreading out the pulses over more bursts (as described above). In addition, it was observed that switching the electrode polarity for a monophasic waveform significantly reduced microbubble formation. This is likely because monophasic waveforms (without switching electrode polarity) typically result in significantly more charge accumulation on the assigned electrode than biphasic waveforms. Accordingly, implementing electrode polarity switching for monophasic waveforms significantly reduces that charge accumulation, reducing microbubble formation. Thus, monophasic waveforms with electrode polarity switching advantageously reduce charge accumulation (like biphasic waveforms), with a longer time between the polarity switching (relative to biphasic waveforms), which may improve lesion formation.

### Electrode Multiplexing

As described above, electrode multiplexing may also impact microbubble formation. To evaluate this impact, microbubble formation was evaluated for different energization schemes.

Specifically, microbubble formation was evaluated for the first energization scheme (described above) and a third energization scheme. In the first energization scheme, multiplexing is implemented, as pulses are delivered over four different electrode channel windows, with two pairs of electrodes delivering waveforms within each window. In the third energization scheme, no multiplexing is implemented. For example, for catheter assembly 250 (shown in FIGS. 3B and 3C) in the third energization scheme, all eight electrodes are fired within a single electrode channel window (e.g., lasting 125 μs). Accordingly, the third energization scheme may be referred to as a simultaneous configuration.

It was observed that the simultaneous configuration (i.e., no multiplexing) facilitates reducing microbubble formation. Further, the microbubble formation may be further reduced by spreading out the pulses over a larger period of time, as described above.

### Grounding Electrode Arrangements

As noted above, grounding electrode arrangements may also impact microbubble formation. For example, as noted above, the decay of accumulated charge on the electrodes effects electrolysis and microbubble formation. Accordingly, techniques may be implemented to remove accumulated charge more rapidly after each pulse application.

For example, in one embodiment, a ground electrode is positioned near the therapy site to reduce charge buildup. In another embodiment, the pulse generating electrodes are periodically switched to ground, using switching circuitry, to reduce charge buildup (e.g., after each pulse, after a group of pulses, or after a burst). These techniques may achieve a reduction in microbubble formation without spreading the pulses over a larger number of bursts, which may reduce overall therapy time.

### Electrode Surface Area and Polarity

As noted above, electrode surface area (and associated polarity) may also impact microbubble formation. To evaluate this impact, microbubble formation was evaluated for various electrode surface areas and polarities.

Specifically, it was observed that delivering a negative monophasic waveform (including one or more negative pulses) between a first, larger electrode set at a positive polarity and a second, smaller electrode set at a negative polarity facilitates reducing microbubble formation. It appears that the larger surface area of the first electrode creates less electrolysis, and thus reduces microbubble formation. This result may also be achieved by delivering a positive monophasic waveform between a first, larger electrode set at a negative polarity and a second, smaller electrode set at a positive polarity.

Notably, delivering a negative monophasic waveform between a first, larger electrode set at a positive polarity and a second, smaller electrode set at a negative polarity results in microbubble formation levels similar to that observed for biphasic waveforms. Further, lesion performance for the monophasic waveform does not appear to be impacted. Thus, this configuration has the advantages of both an increased lesion size (e.g., due to larger reach of the electric field, and because it is a monophasic waveform) and reduced microbubble formation (comparable to that of a biphasic waveform).

With this in mind, catheter designs may be modified accordingly to reduce microbubble formation. For example, consider catheter assembly 250 (shown in FIGS. 3B and 3C). In one embodiment, electrodes that are set to a negative voltage in the energization scheme (e.g., E2, E4, E6, and E8 in the first energization scheme) each have a smaller surface area than electrodes that are set to a positive voltage in the energization scheme while delivering a negative monophasic pulse (e.g., E1, E3, E5, and E7 in the first energization scheme). Of course, this concept could also be extended to any other suitable catheter embodiments (e.g., those shown in FIGS. 2 and 3A).

In another embodiment, where all electrodes have the same surface area, multiple electrodes may be activated together to achieve a larger effective surface area. For example, in catheter assembly 250, electrodes E2 and E3 may both be set to a positive voltage, and electrode E1 may be set to a negative voltage, while delivering a negative monophasic pulse. When electrodes E2 and E3 are fired at the same time (at the positive voltage), they effectively function as a single cathode that is larger than the negative voltage E1, while delivering a negative monophasic pulse. Those of skill in the art will appreciate that other, similar techniques may be implanted in other catheter assemblies. For example, the first and second electrodes (with the first electrode having a larger surface area than the second electrode) may both be included on a linear catheter. Again, this concept could also be extended to any other suitable catheter embodiments (e.g., those shown in FIGS. 2 and 3A).

The systems and methods described herein are directed to electroporation devices. An electroporation system includes a catheter comprising a plurality of electrodes, and a pulse generator coupled to the catheter, the pulse generator configured to generate a waveform to be delivered using at least one of the plurality of electrodes. The waveform includes a plurality of bursts, each burst including a plurality of loops, and each loop including a plurality of pulses, wherein each of the plurality of pulses has a pulse width of 3 microseconds (μs) or less, wherein each burst includes no more than ten loops, wherein the plurality of bursts include at least ten bursts, and wherein the pulse widths, number of loops per burst, and number of bursts facilitate reducing microbubble formation.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. An electroporation system comprising:
a catheter comprising a plurality of electrodes; and
a pulse generator coupled to the catheter, the pulse generator configured to generate a waveform to be delivered using at least one of the plurality of electrodes, the waveform including:
a plurality of bursts, each burst including a plurality of loops, and each loop including a plurality of pulses, wherein each of the plurality of pulses has a pulse width of 3 microseconds (μs) or less, wherein each burst includes no more than ten loops, wherein the plurality of bursts include at least ten bursts, and wherein the pulse widths, number of loops per burst, and number of bursts facilitate reducing microbubble formation.

2. The electroporation system in accordance with claim 1, wherein the plurality of pulses include both positive and negative pulses.

3. The electroporation system in accordance with claim 1, wherein the plurality of pulses include:
a first pulse delivered between a first electrode of the plurality of electrodes and a second electrode of the plurality of electrodes, wherein the first electrode is set to a positive voltage for delivery of the first pulse; and
a second pulse delivered between the first electrode and third electrode of the plurality of electrodes, wherein the first electrode is set to a negative voltage for delivery of the second pulse.

4. The electroporation system in accordance with claim 1, wherein each of the plurality of pulses has a pulse width of 1 μs or less.

5. The electroporation system in accordance with claim 1, wherein the plurality of bursts includes at least fifteen bursts, and wherein each burst includes no more than five loops.

6. The electroporation system in accordance with claim 1, wherein the plurality of bursts includes at least twenty bursts, and wherein each burst includes no more than three loops.

7. A pulse generator for use with an electroporation system, the pulse generator configured to be coupled to a catheter including a plurality of electrodes and configured to generate a waveform to be delivered using at least one of the plurality of electrodes, the waveform including:
a plurality of bursts, each burst including a plurality of loops, and each loop including a plurality of pulses, wherein each of the plurality of pulses has a pulse width of 3 microseconds (μs) or less, wherein each burst includes no more than ten loops, wherein the plurality of bursts include at least ten bursts, and wherein the pulse widths, number of loops per burst, and number of bursts facilitate reducing microbubble formation.

8. The pulse generator in accordance with claim 7, wherein the plurality of pulses include both positive and negative pulses.

9. The pulse generator in accordance with claim 7, wherein the plurality of pulses include:
a first pulse delivered between a first electrode of the plurality of electrodes and a second electrode of the plurality of electrodes, wherein the first electrode is set to a positive voltage for delivery of the first pulse; and
a second pulse delivered between the first electrode and third electrode of the plurality of electrodes, wherein the first electrode is set to a negative voltage for delivery of the second pulse.

10. An electroporation system comprising:
a catheter comprising a plurality of electrodes; and
a pulse generator coupled to the catheter, the pulse generator configured to generate a waveform to be delivered using at least one of the plurality of electrodes, the waveform including at least a first pulse delivered between a first electrode of the plurality of electrodes and a second electrode of the plurality of electrodes; and
a grounding electrode arrangement configured to discharge, from the first and second electrodes, charge accumulated on the first and second electrodes during delivery of the first pulse.

11. The electroporation system in accordance with claim 10, wherein the grounding electrode arrangement comprises a ground electrode positioned proximate the first and second electrodes.

12. The electroporation system in accordance with claim 10, wherein the grounding electrode arrangement comprises switching circuitry that is configured to periodically switch the first and second electrodes to ground.

13. The electroporation system in accordance with claim 10, wherein the waveform is a biphasic waveform.

14. The electroporation system in accordance with claim 10, wherein the waveform is a monophasic waveform.

15. The electroporation system in accordance with claim 10, wherein the waveform includes a plurality of bursts, each burst including a plurality of loops, and each loop including a plurality of pulses, wherein each of the plurality of pulses has a pulse width of 3 microseconds (μs) or less, wherein each burst includes no more than ten loops, wherein the plurality of bursts include at least ten bursts, and wherein the pulse widths, number of loops per burst, and number of bursts facilitate reducing microbubble formation.

16. The electroporation system in accordance with claim 15, wherein the plurality of pulses include both positive and negative pulses.

17. The electroporation system in accordance with claim 15, wherein each of the plurality of pulses has a pulse width of 1 μs or less.

18. The electroporation system in accordance with claim 15, wherein the plurality of bursts includes at least fifteen bursts, and wherein each burst includes no more than five loops.

19. The electroporation system in accordance with claim 15, wherein the plurality of bursts includes at least twenty bursts, and wherein each burst includes no more than three loops.

20. The electroporation system in accordance with claim 15, wherein each of the plurality of pulses has a pulse width of 2 μs or less.
